(19) United States
(12) Patent Application Publication
(10) Pub. No.: US 2019/0117528 A1
(43) Pub. Date: Apr. 25, 2019

(54) AEROSOL GEL COMPOSITION

(71) Applicant: PZ Cussons (International) LTD, Manchester (GB)

(72) Inventors: Paul Heffernan, Oakmere (GB); Phillip Shaw, Bowdon (GB); Meiki Wu, Droylsden (GB); Craig Davies, Alsager (GB)

(21) Appl. No.: 16/096,955

(22) PCT Filed: Mar. 16, 2017
(86) PCT No.: PCT/GB2017/050726

§ 371 Date: Oct. 26, 2018

(30) Foreign Application Priority Data
Apr. 26, 2016 (GB) 1607199.5

(51) Int. Cl. A61K 8/04; A61Q 19/10; A61K 8/81; A61K 8/31; A61K 8/46; A61K 8/41

(52) U.S. Cl. CPC A61K 8/042; A61Q 19/10; A61K 8/046; A61K 2800/87; A61K 8/31; A61K 8/463; A61K 8/41; A61K 8/8152

(57) ABSTRACT

The present invention relates to a self-foaming or post-foaming gel or cream composition, comprising surface active agents and polymer, plus a post-foaming agent packaged in an aerosol or other container. The composition is milder than a typical soap based system, and delivers acceptable performance at a cost below that of equivalently performing systems based upon synthetic surfactants.

### CROSS REFERENCE TO RELATED APPLICATIONS

This application is a National Stage application of PCT/GB2017/050726, filed Mar. 16, 2017, which claims the benefit of Great Britain Application No. 1607199.5, filed Apr. 26, 2016, both of which are incorporated by reference in their entirety herein.

### FIELD

The present invention relates to a self-foaming or post-foaming gel or cream composition which can be dispensed by an aerosol, the composition comprising surface active agents, a polymer, plus a post-foaming agent, and being packaged in an aerosol or other container. The composition is milder to a person's skin than a typical soap based system, and delivers acceptable performance at a cost below that of equivalently performing systems based upon synthetic surfactants.

### BACKGROUND

Aerosol post foaming gels and creams are widely used for a variety of personal care applications. However, they are most commonly employed for shaving preparations. Typically these use low cost, but potentially harsh, fatty acid soap based formulae, or more expensive, but potentially milder, synthetic surfactant based systems. These synthetic surfactant based systems tend to be more expensive as they usually require high levels of surfactants and/or high levels of polymeric thickeners to achieve the necessary structure required. They can also demonstrate instability and incompatibility in combination with common packaging materials and can be difficult to process due to the viscous nature of the formulations.

Post foaming aerosol gels were originally developed for shaving products (U.S. Pat. No. 3,541,581A by Monson), and originally used metallic soaps to assist in generating an acceptable lather. Soap free systems were then developed to mitigate the harsh skin feel and drying associated with soap based systems (U.S. Pat. No. 4,772,427 by Dawson et al; and US2008031843 by Aubert et al). Post foaming gels have also been previously described as delivering excellent sensory benefits when used in showers (US2012263668 by Cowan et al; US2008261842 by Hall & Yaqub; US20040258643 by Yaqub et al; WO2011/042759 by Cowan et al).

It is known from US2008261842 that the gel structure formed can be reliant on the level of isopentane present in the formulation, and that low levels of isopentane may cause an instability in the gel structure. Over time, instability of the gel may occur as the affinity for certain polymers within the aerosol packaging for isopentane may cause a localised decrease of the isopentane level within the filled product, causing localised destructuring of the gel and a “wet patch” that is dispensed from the pack upon actuation.

U.S. Pat. No. 4,726,944 by Osipow et al describes the use of amine oxide surfactants in post foaming gel systems; however, they are used in combination with an aqueous soap solution and free fatty acids and as such are harsher to the skin, and rely upon the gel structure being generated by the soap and fatty acid components of the mixture combined with a water soluble gum. It also proposes that the formulation be packaged in a non-aerosol format, however in reality this is impractical due to the pressures generated by the volatile hydrocarbon foaming agent at elevated temperatures.

GB2236760 by Mann & Garien describes the use of in situ neutralised polymers in the formation of a post foaming gel. However, this invention is focused specifically on the delivery of a shaving preparation and uses a specific class of polymer, unrelated to our invention, and does not rely upon the neutralisation of the polymer by a surface active agent within the formula to develop its structure.

WO2004004678 by Kuether & Rohde describes the use of starch derived polymers in combination with an anionic surfactant system.

There is therefore a need for a personal cleansing composition which is able to overcome one or more of the above problems, and is able to achieve an acceptably firm and stable gel while using a lower level of polymer, and which provides a consumer acceptable product at a lower cost.

### SUMMARY

Therefore, in accordance with the present invention there is provided a post-foaming composition comprising:
a) water;
b) one or more anionic surfactants in an amount of about 2 to about 20% by weight of the composition;
c) one or more amine oxide surfactants in an amount of about 1 to about 5% by weight of the composition;
d) a hydrophobically modified alkali-swellable acrylic emulsion (HASE) polymer in an amount of about 0.5 to about 3% polymer solids by weight of the composition; and
e) a foaming agent comprising a non-polar saturated aliphatic hydrocarbon having from 3 to 6 carbon atoms;
   wherein the composition is capable of suspending water insoluble or partially water soluble substances in the form of particles or liquid droplets.

The concept of ‘alkali-swellable’ polymers is well known in the polymer technical field, and as such will be familiar to, and understood by, a person skilled in the art. Such HASE polymers are also commercially available, for example under the ACULYN® trade name from Dow Chemicals, or under the Rheovis® and Latekoll® trade names from BASF.

In their commercially available forms, the polymers (which are acidic in their nature) are coiled up tightly and dispersed in an aqueous medium, essentially as insoluble particles. In the presence of an alkali, the acidic polymer is neutralized, and expands due to negative charge repulsion. The particles of polymer are therefore said to swell, with water filling up the spaces. Not all synthetic anionic polymers swell in the presence of alkalis, for example the type used as opacifying agents (e.g. styrene/acrylates copolymer) are designed to remain in a compact state.

When HASE polymers/rheology modifiers are neutralized, they become anionically charged and water-soluble. When HASE polymers dissolve, they swell due to charge-to-charge repulsion and thicken instantly. As in the case of cross-linked alkali-swellable emulsion (ASE) polymers, structure is built as the critical concentration is exceeded, but in the case of the HASE rheology modifiers, it is by polymer chain entanglement, in conjunction with association of the extended hydrophobic groups on the polymer backbone with each other and other hydrophobic groups in a formulation.

### DETAILED DESCRIPTION

The invention uses a combination of an anionic surfactant combined with both of an amine oxide surfactant and a HASE polymer. The resulting combination is then blended with a post foaming agent and packaged in to a bi-compartmental or other suitable aerosol system.

The combination of the anionic surfactant and amine oxide surfactant provides a relatively mild and low cost cleansing mixture. It is milder than a typical soap based system and delivers acceptable performance at a cost that is typically less than equivalently performing systems based upon synthetic surfactants. A key driver of this benefit is that only relatively low levels (5-15%) of overall surfactant are required to create a consumer acceptable product.

When using this surfactant mixture in combination with the HASE polymer, it is possible to form a gel of sufficient viscosity and texture to be used as an aerosol post foaming gel. At this stage the gel has a poor degree of optical clarity.

When the gel is then mixed with a suitable post foaming agent, such as isopentane, and pressurised, the gel structure undergoes a transition to a clear (transparent/translucent) gel. This is visually comparable to other post foaming aerosol gels whilst only using a low level of surfactant and delivering in use attributes equivalent or superior to comparable current products.

The composition is packaged in to an aerosol container or any such container that can dispense a post-foaming gel. This may be a bi-compartmental aerosol container. A bi-compartmental aerosol container than can be used to contain the composition of the invention may take the form of a bag in can, bag on valve, piston, elasticated bladder or other barrier system. Application of an external pressure to the formulation-containing compartment enables the formulation to be dispensed on opening a valve, actuated by the consumer during product usage.

The pressure must be sufficient to prevent the post-foaming agent transforming to a gaseous state inside the formulation compartment, even at elevated temperature. This may be applied either by mechanical means (such as an elasticated bladder system) or via an ‘external’ propellant that resides in the space between the formulation compartment and the outer container. In the case of the latter the external propellant gas may be either a liquefied or compressed gas, such as a C1-C4 hydrocarbon, a fluorocarbon, nitrous oxide, compressed air or nitrogen.

Typically, the compositions of the invention are substantially free of soap.

Unlike existing compositions, the composition of the invention does not rely upon the surfactant-isopentane interaction in order to maintain its primary structure, instead relying upon the presence of the HASE polymer. This unexpected discovery makes the new formula more stable than currently known formulations.

Additionally, as the composition of the invention does not rely upon high levels of surfactant or soap to develop an acceptably firm gel structure, the required level of surfactant to produce an acceptably firm gel is lower (about 5 to about 15% surfactant) than that required for other compositions. The composition of the invention is thus able to achieve a consumer acceptable product at a lower cost than other known formulations.

In detail, the present invention, while not wishing to be bound by any particular theory, is explained below:

The inventors have found that mixing a pH neutral solution of an anionic surfactant with a pH neutral solution of an amine oxide produces an unexpected rise in pH of the aqueous medium (from 7.0 to about 9.0) Amine oxides are non-ionic surfactants at neutral pH but with a strong dipole across the N—O bond. It is reasonable to hypothesise that this strong dipole is capable of interacting with anionic surfactants and surrounding water molecules which may account for the observed pH rise. Further, the inventors have found that inclusion of a particular type of polymer (Hydrophobically modified Alkali Swellable Emulsion polymer, herein referred to as HASE polymer for short) produces a 3-way interaction between the polymer and the two surfactant types which gives an unexpectedly large increase in viscosity.

This effect is exploited in the present invention in two ways. Firstly, the rise in pH from the surfactant interaction described earlier can be used to neutralise the acidic HASE polymer which would otherwise require the use of a basic neutralising agent such as sodium hydroxide. Secondly, the inventors have found that resulting anionic-amine oxide surfactant mixture interacts strongly with the HASE polymer. This enables the use of lower polymer inclusion levels than those which would normally be required, and this system is capable of providing a very high zero-shear viscosity to enable suspension of particulates and oil droplets. It is believed that this type of structurisation has not been used before. The surfactant interaction does not create lamellar structures as taught in the prior art; rather, it is believed that the pairing of the two surfactant molecules creates a more hydrophobic (and larger) entity that interacts more strongly with the hydrophobic appendages on the HASE polymer, building a stronger surfactant-polymer network.

The pairing of the surfactant molecules in this manner is able to reduce irritancy compared with using the anionic surfactant alone by: a) reducing the amount of free monomers in solution; b) the anionic charge neutralisation reducing its binding affinity to skin proteins; and c) the larger paired surfactant entities being less able to penetrate the skin. Evidence of this is provided in Surfactants in Cosmetics (Surfactant Science Series volume 68, published by CRC press), 2^{nd} edition, page 405, under the heading “Surfactant interactions that counter skin irritation”, where data is provided using an in-vitro based method based on swelling of isolated stratum corneum tissue. This shows that the degree of swelling, as measured by % increase in hydrated length, was reduced from 46.1% (1% sodium lauryl sulfate (SLS) only) to just 7.7% for a solution containing 1% SLS+1% lauryldimethylamine oxide (LDAO). The result for a solution of 1% SLS+1% cocamidopropyl betaine (a commonly used amphoteric surfactant well known for reducing irritancy of anionics when used in combination) was 21.0%, a significant reduction but not as dramatic as in the case of LDAO.

Furthermore, the inventors have also found that the resulting compositions have advantageous sensory qualities when used for personal washing, in particular producing a voluminous yet creamy lather and a pleasant skin-feel during the washing process which persists as a conditioned feel after washing.

A further advantage of this novel approach is that it uses common surfactants, and is thus cost-effective. The resulting compositions have a desirably thick but shear-thinning gel texture. This texture is not dependent on the presence of oils, but is flexible in its ability to allow the stable incorporation of oils from zero up to at least 50%. It is also easy to manufacture, has good lathering characteristics and has been found to leave the skin feeling soft and smooth.

### HASE Polymer Constituent:

Hydrophobically modified alkali-swellable emulsion (HASE) thickeners are the associative analogue to alkali-swellable emulsion (ASE) synthetic thickeners which are produced commercially by emulsion polymerisation. Like the ASE thickeners, HASE polymers have ‘backbones’ made from methacrylic acid and various acrylate/methacrylate alkyl ester monomers. However, unlike typical ASE types, HASE polymers contain hydrophobic “pendants” which are commonly attached via polyethylene oxide chains. Thickening is achieved by 2 mechanisms: firstly, upon addition of an alkaline neutralising base (for example an alkali metal hydroxide) the —COOH moieties along the backbone become ionised to —COO⁻ carboxyl groups which cause the polymer chain to expand by electrostatic repulsion and become soluble in water, changing from a milky low viscosity dispersion to a transparent gel. Secondly, the hydrophobic pendant groups are able to build associations with each other and also with surfactants, oil droplets and any other dispersed particulates. This creates a network structure that is accompanied by significant viscosity build.

Suitable HASE polymers for use in the present invention are anionic in nature upon neutralisation. They are typically supplied in the form of an aqueous liquid emulsion (which has an un-neutralised, acidic pH) form with hydrophobic appendages which make them ‘associative’—i.e. they can associate with other formulation ingredients, such as surfactants, oils or particles.

Suitable HASE polymers include, but are not limited to, those which are commercially available from Rohm & Haas under the Aculyn trade name, such as Aculyn 22 (acrylates/steareth-20 methacrylate copolymer), Aculyn 28 (acrylates/beheneth-25 methacrylate copolymer) and Aculyn 88 (acrylates/steareth-20 methacrylate crosspolymer). Another option is Synthalen W2000 from 3V (acrylates/palmeth-25 acrylate copolymer).

The HASE polymer is present in an amount of about 0.5 to about 2.5% by weight (as polymer solids) of the total composition, typically from about 1 to about 2% by weight.

### Anionic Surfactant Constituent:

Suitable anionic surfactants for use in the composition of the invention include, but are not limited to, alkyl sulfates (e.g. sodium lauryl sulfate or sodium cocosulfate); ethoxylated alkyl sulfates (e.g. sodium laureth sulfate or sodium trideceth sulfate); alpha olefin sulfonates; alkyl alkoxy carboxylates; acyl isethionates (e.g. sodium cocoyl isethionate); acyl sarcosinates (e.g. sodium cocoyl sarcosinate); sulfosuccinates (e.g. disodium laureth sulfosuccinate); or acyl glutamates such as sodium cocoyl glutamate. Alternative counter ions may be used in place of sodium, such as potassium, ammonium, triethanolammonium (TEA), or monoisopropylammonium (MIPA). According to one embodiment, the anionic surfactant includes, or is, sodium laureth sulfate.

The one or more anionic surfactants are present in an amount of about 2 to about 20% by weight of the composition; typically from about 3 to about 15% by weight, more typically from about 4 to about 12% by weight, still more typically from about 5 to about 10% by weight.

### Amine Oxide Constituent:

Suitable amine oxide surfactants for use in the composition of the invention include, but are not limited to, alkamine oxides containing at least one long hydrocarbon chain containing at least eight carbon atoms.

One such class of amine oxides is the alkyl di(lower alkyl) amine oxides, wherein the alkyl group therein contains from 8 to 22, and preferably about 10 to about 16, carbon atoms, and can be straight or branched chain, saturated or unsaturated. The lower alkyl groups in the alkyl di(lower alkyl) amine oxides contain from 1 to 7 carbon atoms, and typically are methyl, ethyl, propyl or butyl, more typically methyl or ethyl. Specific examples include, but are not limited to, lauryl dimethyl amine oxide, myristyl dimethyl amine oxide, dimethyl cocoamine oxide, dimethyl (hydrogenated tallow)amine oxide, myristyl/palmityl dimethyl amine oxide, myristyl/lauryl dimethyl amine oxide, cetyl dimethyl amine oxide, stearyl dimethyl amine oxide, and myristyl/cetyl dimethyl amine oxide. These alkamine oxides have a general structural formula:

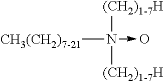

Another class of useful amine oxides for use in the present invention includes alkyl di(hydroxy lower alkyl)amine oxides in which the alkyl group contains from 8 to 22, and preferably about 10 to about 16 carbon atoms, and can be straight or branched chain, saturated or unsaturated. The lower alkyl groups in the alkyl di(lower alkyl) amine oxides contain from 1 to 7 carbon atoms, and typically are methyl, ethyl, propyl or butyl, more typically methyl or ethyl. Specific examples, include, but are not limited to, bis(2-hydroxyethyl)cocoamine oxide, and bis(2-hydroxyethyl)stearylamine oxide. These alkamine oxides have a general structural formula:

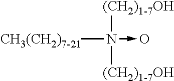

Additional useful amine oxides for use in the present invention are termed alkamidopropyl di(lower alkyl)amine oxides in which the alkyl group contains from 8 to 22, and preferably about 10 to about 16 carbon atoms, and can be straight or branched chain, saturated or unsaturated. The lower alkyl groups in the alkyl di(lower alkyl) amine oxides contain from 1 to 7 carbon atoms, and typically are methyl, ethyl or propyl, more typically methyl. Non-limiting examples thereof are cocoamidopropyl dimethyl amine oxide and lauramidopropyl dimethyl amine oxide. These alkamine oxides have a general structural formula:

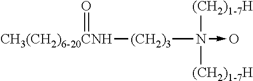

The above classes of alkamine oxide surfactants contain one or more C8-C22 alkyl groups selected from, for example, octyl, decyl, undecyl, lauryl, tridecyl, myristyl, cetyl, stearyl, isostearyl, oleyl, and mixtures thereof. Non-limiting examples of amine oxide surfactants include, but are not limited to, decyl dimethylamine oxide, lauryl dimethylamine oxide, stearyl dimethylamine oxide, oleyl dimethylamine oxide, coco dihydroxyethylamine oxide, cetyl N,N-dihydroxyethylamine oxide, oleyl N,N-dihydroxyethylamine oxide, cocamine oxide, cocamidopropylamine oxide, lauramidopropylamine oxide, oleamine oxide, oleamidopropylamine oxide, wheat germamidopropylamine oxide, isostearamido-propylamine oxide, stearamine oxide, stearamido-propylamine oxide, decylamine oxide, dihydroxyethyl C8-C18 alkoxypropylamine oxide, myristamidopropylamine oxide, myristamine oxide, palmitamidopropylamine oxide, palmitamine oxide, undecylenamidopropylamine oxide, and mixtures of any two or more thereof. Preferred alkamine oxide surfactants are the alkyl di(lower alkyl)amine oxides in which the alkyl group contains about 12 to about 16 carbon atoms, including lauramine oxide, myristamine oxide, cocamine oxide, cetamine oxide, and mixtures of any two or more thereof. According to one embodiment of the invention, the amine oxide surfactant comprises, or is, lauramine oxide.

The one or more amine oxide surfactants are present in an amount of about 1 to about 10% by weight of the composition; typically from about 1 to about 8% by weight, more typically from about 1.5 to about 7% by weight, still more typically from about 2 to about 5% by weight.

### Foaming Agent Constituent

Typically, the foaming agent comprises one or more compounds selected from isopentane, n-pentane, neopentane, isobutane or n-butane, in an amount of between about 2% and about 20%, more typically between about 5% and about 15%, by weight of the composition.

By ‘post-foaming’, it is meant herein that the composition contains a volatile foaming agent that transforms from a liquid or gelled state (by being dissolved, emulsified or otherwise entrapped in the formulation matrix) to a gaseous state after dispensation from its retail packaging, and during intended usage, to generate foam internally within the applied formulation, by virtue of this change of physical state of said agent. Unlike conventional cleansing products, such formulations are therefore not reliant on entrainment of atmospheric air by mechanical action to generate foam.

The foaming agent is typically present in an amount of about 1 to about 20% by weight of the composition; more typically from about 2.5 to about 16% by weight, more typically from about 5 to about 15% by weight, still more typically from about 8 to about 12% by weight.

According to another embodiment of the invention, the composition may further contain one or more of the following components:

Oils (including vegetable triglycerides, mineral oil, petrolatum, hydrocarbons, silicones, plant derived butters, esters, essential oils, compounded fragrance oils), natural or synthetic scrub particles (e.g. polyethylene, silica, rice bran, loofah, pumice, diatomaceous earth, pearlite, ground fruit or nut ‘stones’ or shells), visual effect agents (e.g. wax particles, polymeric opacifiers, inorganic ‘shimmers’), humectants (e.g. glycerol, sorbitol, glycols, urea, amino acids, lactates), cationic conditioning agents (such as cationic guar or cassia gum derivatives, Polyquaternium-7, Polyquaternium-39, Polyquaternium-10 etc), preservatives, colourants, chelating agents and pH adjusters (acids or bases). The formulation may optionally include a clay based rheology modifier such as a bentonite, hectorite, montmorillonite or synthetic clay such as Laponite (Rockwood Products) which is available in various grades.

The various components of the composition of the invention are added in their respective amounts as desired; the balance of the composition to 100% is water.

The composition of the invention may further contain one or more ingredients selected from emollients, moisturizers, conditioners, skin conditioners, hair conditioners, vitamins, pro-vitamins, antioxidants, free-radical scavengers, abrasives, dyes, hair coloring agents, bleaching agents, UV absorbers, anti-UV agents, antibacterial agents, antifungal agents, melanin regulators, tanning accelerators, depigmenting agents, skin lightening agents, skin-coloring agents, liporegulators, weight-reduction agents, anti-acne agents, antiseborrhoeic agents, anti-ageing agents, anti-wrinkle agents, keratolytic agents, anti-inflammatory agents, anti-acne agents, antibiotics, anti-inflammatory agents, botanical extracts, imidazoles, refreshing agents, cicatrizing agents, vascular-protection agents, agents for the reduction of dandruff, seborrheic dermatitis, or psoriasis, shale oil and derivatives thereof, anti-psoriasis agents, corticosteroids depilating agents, agents for combating hair loss, reducing agents for permanent-waving, reflectants, essential oils and fragrances.

The pH of the invention is typically in the range of about 6.0 to about 10.0, and more typically in the range of about 6.5 to about 8.0. The visual appearance of the composition in the absence of suspended matter is a transparent or translucent gel.

Such post-foaming compositions are suitable for use as personal cleansing compositions, such as a body wash, facial wash or hand wash, for washing hair, or as shaving gels, or the delivery of other benefit agents for skin or hair as previously described.

The composition of the invention is provided in a packaged form to consumers. The package is typically an aerosol container or any such container that can dispense a post foaming gel; more typically it is a bi-compartmental aerosol container where the composition is separated from a propellant gas by a bag, piston or other barrier. The propellant gas may be either a liquefied or compressed gas.

Also provided in accordance with the present invention is a method of manufacturing a personal cleansing composition comprising:
a) water;
b) one or more anionic surfactants in an amount of about 2 to about 20% by weight of the composition;
c) one or more amine oxide surfactants in an amount of about 1 to about 10% by weight of the composition;
d) a hydrophobically modified alkali-swellable acrylic emulsion (HASE) polymer in an amount of about 0.5 to about 3% polymer solids by weight of the composition; and
e) a foaming agent comprising a non-polar saturated aliphatic hydrocarbon having from 3 to 6 carbon atoms;
   wherein the composition is capable of suspending water insoluble or partially water soluble substances in the form of particles or liquid droplets,
   the method comprising the steps of:
i) loading an amount of water into a suitable vessel;
ii) adding a HASE polymer, one or more anionic surfactants, and one or more amine oxide surfactants, sequentially in any order, with sufficient mechanical stirring to ensure that each material is fully dispersed before adding the next;
iii) continuing mixing until a uniform consistency is achieved; and
iv) adding a foaming agent

Optionally, one or more cationic conditioning agents may also be added into the water, before or after the addition of the HASE polymer and surfactants.

An exemplary, but non-limiting, method in accordance with the invention comprises the following steps:
i) dissolving one or more anionic surfactants in an amount of water;
ii) adding a HASE polymer emulsion, mixing until the polymer is substantially dispersed in the one or more anionic surfactants;
iii) adding one or more amine oxide surfactants;
iv) mixing until substantially uniform consistency is achieved; and
v) adding a foaming agent.

Optionally, one or more cationic conditioning agents may also be added into the water, before the addition of the one or more anionic surfactants to the amount of water. Also, further optional minor ingredients, such as chelating agents, preservatives, fragrances, humectants, and further materials to be suspended if required (e.g. beads, scrubs, etc.), may also be added after the addition of the HASE polymer emulsion.

The formulation remains unstructured and generally of low viscosity until the addition of amine oxide, at which point the structure develops as the surfactants and polymer interact with each other. The final ingredient (hydrocarbon foaming agent) is added with appropriate mixing to ensure the final mixture is homogeneous.

There is also provided a further step for the method of the invention, which comprises packaging the composition into an aerosol container or any such container that can dispense a post foaming gel.

Also provided in accordance with the present invention is a use of a composition comprising:
a) water;
b) one or more anionic surfactants in an amount of about 2 to about 20% by weight of the composition;
c) one or more amine oxide surfactants in an amount of about 1 to about 10% by weight of the composition; and
d) a hydrophobically modified alkali-swellable acrylic emulsion (HASE) polymer in an amount of about 0.5 to about 2% by weight polymer solids by weight of the composition; and
e) a foaming agent comprising a non-polar saturated aliphatic hydrocarbon having from 3 to 6 carbon atoms.
   wherein the composition is capable of suspending water insoluble or partially water soluble substances in the form of particles or liquid droplets, as a personal cleansing composition,

The formulation remains unstructured and generally of low viscosity until the addition of amine oxide, at which point the structure develops as the surfactants interact with each other and then with the polymer. The final ingredient (hydrocarbon foaming agent) is added with appropriate mixing to ensure the final mixture is homogeneous.

Also provided in accordance with the present invention is a use of a composition comprising:

a) water;

b) one or more anionic surfactants in an amount of about 2 to about 20% by weight of the composition;

c) one or more amine oxide surfactants in an amount of about 1 to about 10% by weight of the composition; and

d) a hydrophobically modified alkali-swellable acrylic emulsion (HASE) polymer in an amount of about 0.5 to about 2% by weight polymer solids by weight of the composition; and

e) a foaming agent comprising a non-polar saturated aliphatic hydrocarbon having from 3 to 6 carbon atoms

as a vehicle for delivery of one or more ingredients selected from emollients, moisturizers, conditioners, skin conditioners, hair conditioners, vitamins, pro-vitamins, antioxidants, free-radical scavengers, abrasives, dyes, hair coloring agents, bleaching agents, UV absorbers, anti-UV agents, antibacterial agents, antifungal agents, melanin regulators, tanning accelerators, depigmenting agents, skin lightening agents, skin-coloring agents, liporegulators, weight-reduction agents, anti-acne agents, antiseborrhoeic agents, anti-ageing agents, anti-wrinkle agents, keratolytic agents, anti-inflammatory agents, anti-acne agents, antibiotics, anti-inflammatory agents, botanical extracts, imidazoles, refreshing agents, cicatrizing agents, vascular-protection agents, agents for the reduction of dandruff, seborrheic dermatitis, or psoriasis, shale oil and derivatives thereof, anti-psoriasis agents, corticosteroids depilating agents, agents for combating hair loss, reducing agents for permanent-waving, reflectants, essential oils and fragrances to skin or hair.

The present invention will be further described with reference to the following examples, which are intended to be illustrative only, and in no way limiting upon the scope of the invention.

### Examples

TABLE 1

| | | Formulations | | | | | | |
|---|---|---|---|---|---|---|---|---|
| Ingredients | Note | A | B | C | D | E | F | G |
| Sodium Laureth Sulfate | 1 | 7.28 | 7.28 | | | 6.83 | | |
| Sodium Lauroyl Sarcosinate | 2 | | | | | | | |
| Sodium Cocoyl Isethionate | 3 | | | 6.83 | | | | |
| Sodium Lauroyl Methyl | 4 | | | | 3.41 | | | |
| Isethionate | | | | | | | | |
| Disodium Laureth | 5 | | | | | | 6.83 | |
| Sulfosuccinate | | | | | | | | |
| Disodium Cocoyl Glutamate | 6 | | | | | | | 6.83 |
| Lauramine Oxide | 7 | | | | | | | |
| Lauramine/Myristamine Oxide | 8 | 1.82 | | 2.28 | 1.14 | 2.28 | 2.28 | 2.28 |
| Cocamine Oxide | 9 | | 1.82 | | | | | |
| Lauric/Myristic | 10 | | | | | | | |
| Amidopropylamine Oxide | | | | | | | | |
| Acrylates/Steareth-20 | 11 | 0.86 | 1.10 | 1.30 | 0.92 | | 1.18 | 1.18 |
| methacrylate crosspolymer | | | | | | | | |
| Acrylates/Palmeth-25 acrylate | 12 | | | | | | | |
| crosspolymer | | | | | | | | |
| Acrylates/Beheneth-25 | 13 | | | | | 1.18 | | |
| methacrylate copolymer | | | | | | | | |
| Sodium Lithium Magnesium | 14 | | 0.18 | 0.36 | | 0.36 | 0.36 | 0.36 |
| Silicate | | | | | | | | |
| Polyquaternium-7 | 15 | 0.16 | | | 0.16 | | | |
| PEG-200 Hydrogenated | 16 | | | | 0.91 | | | |
| Glyceryl Palmate (and) PEG-7 | | | | | | | | |
| Glyceryl Cocoate | | | | | | | | |
| Tetrasodium Glutamate | 17 | 0.09 | 0.09 | 0.09 | 0.09 | 0.09 | 0.09 | 0.09 |
| Diacetate | | | | | | | | |
| Glycerin | 18 | | 0.91 | | | | | |
| DMDM Hydantoin | 19 | 0.18 | 0.18 | 0.18 | 0.18 | 0.18 | 0.18 | 0.18 |
| Fragrance Oil | 20 | 0.72 | 0.64 | 0.79 | 0.91 | 0.72 | 0.72 | 0.72 |
| Visual effect pigment (Mica, | 21 | | | | | | | |
| Titanium dioxide, Tin oxide | | | | | | | | |
| composite) | | | | | | | | |
| Sunflower Oil | 22 | | | | | | | |
| Isopentane | 23 | 9.00 | 9.00 | 9.00 | 9.00 | 9.00 | 9.00 | 9.00 |

TABLE 2

| | | Formulations | | | | | | |
|---|---|---|---|---|---|---|---|---|
| Ingredients | Note | H | I | J | K | L | M | N |
| Sodium Laureth Sulfate | 1 | | 6.83 | 5.00 | 4.46 | 4.78 | 7.28 | 7.28 |
| Sodium Lauroyl Sarcosinate | 2 | 6.83 | | | | | | |
| Sodium Cocoyl Isethionate | 3 | | | | | | | |
| Sodium Lauroyl Methyl | 4 | | | | | | | |
| Isethionate | | | | | | | | |
| Disodium Laureth | 5 | | | | | | | |
| Sulfosuccinate | | | | | | | | |
| Disodium Cocoyl Glutamate | 6 | | | | | | | |
| Lauramine Oxide | 7 | | | | | | 1.82 | |
| Lauramine/Myristamine | 8 | 2.28 | 2.28 | 1.66 | 1.49 | 1.60 | | |
| Oxide | | | | | | | | |
| Cocamine Oxide | 9 | | | | | | | |
| Lauric/Myristic | 10 | | | | | | | 1.82 |
| Amidopropylamine Oxide | | | | | | | | |
| Acrylates/Steareth-20 | 11 | 1.18 | | 1.00 | 1.00 | 1.00 | 0.86 | 1.50 |
| methacrylate crosspolymer | | | | | | | | |
| Acrylates/Palmeth-25 acrylate | 12 | | 1.30 | | | | | |
| crosspolymer | | | | | | | | |
| Acrylates/Beheneth-25 | 13 | | | | | | | |
| methacrylate copolymer | | | | | | | | |
| Sodium Lithium Magnesium | 14 | 0.36 | 0.36 | | | | | 0.18 |
| Silicate | | | | | | | | |
| Polyquaternium-7 | 15 | | | | | | 0.16 | 0.27 |
| PEG-200 Hydrogenated | 16 | | | | | | 0.91 | 0.91 |
| Glyceryl Palmate (and) PEG-7 | | | | | | | | |
| Glyceryl Cocoate | | | | | | | | |
| Tetrasodium Glutamate | 17 | 0.09 | 0.09 | 0.09 | 0.09 | 0.09 | 0.09 | 0.09 |
| Diacetate | | | | | | | | |
| Glycerin | 18 | | | | | | 0.91 | 0.91 |
| DMDM Hydantoin | 19 | 0.18 | 0.18 | 0.18 | 0.18 | 0.18 | 0.18 | 0.18 |
| Fragrance Oil | 20 | 0.72 | 0.72 | 0.72 | 0.72 | 0.72 | 0.64 | 0.64 |
| Visual effect pigment (Mica, | 21 | | | | | 0.10 | | |
| Titanium dioxide, Tin oxide | | | | | | | | |
| composite) | | | | | | | | |
| Sunflower Oil | 22 | | | | | | 10.00 | 10.00 |
| Isopentane | 23 | 9.00 | 9.00 | 5.00 | 15.00 | 9.00 | 9.00 | 9.00 |

### Notes

1 Steol CS 270 (Stepan Company)—supplied as 70% active paste

2 Surfacare L30 (Surfachem)—supplied as 30% active solution

3 Elfan AT84 (Akzo Nobel N.V.)—supplied as a powder

4 Iselux LQ-CLR-SB (Innospec)—supplied as a 35% active solution

5 Rewopol SB FA 30B (Evonik Industries A.G.)—supplied as 30% active solution

6 Plantapon ACG HC (BASF Personal Care)—supplied as 50% active solution

7 Mackamine LO E (Solvay Europe)—supplied as 30% active solution

8 Mackamine LA (Solvay Europe)—supplied as 30% active solution

9 Mackamine CS (Solvay Europe)—supplied as 30% active solution

10 Mackamine LMDO (Solvay Europe)—supplied as 30% active solution

11 Aculyn 88 (The Dow Chemical Company)—supplied as a 29% polymer solids dispersion

12 Synthalen W2000 (3V Sigma)—supplied as a 31% polymer solids dispersion

13 Novethix L-10 (The Lubrizol Corporation)—30% polymer solids

14 Laponite XLS (BYK additives)

15 Mackernium 007B (Solvay Europe)

16 Rewoderm LIS 80 (Evonik Industries A.G.)

17 Dissolvine GL38 (Akzo-Nobel N.V.)

18 Cremer Oleo

19 Microcare DH (Thor Personal Care)

20 Seven Scent

21 Timiron Silk Gold (Merck Performance Materials)

22 Food grade, acquired from retail store

23 Sigma Aldrich

In each of the formulations listed in Tables 1 and 2, the balance was made up with water. All figures refer to % w/w as active substance with the exception of Rewoderm LIS 80 for which the inclusion levels are quoted as supplied.

All of the formulations A-N listed above have a pH in the range of 6.0 to 8.0, are opaque white, and have the consistency of self-supporting creams upon dispensation from a pressurised container. The application of gentle shearing forces (such as by rubbing onto a person's skin) causes a transformation into a dense foam or lather.

Examples A to K are all transparent/translucent gels upon dispensation from a pressurised container.

Example L is a translucent gel with an iridescent visual appearance.

Examples M and N are opaque white compositions with a cream-like appearance.

The visual appearances of the formulations of the examples varies from translucent to opaque, depending on the presence of the suspended water-insoluble substances. The compositions of the invention allow a person skilled in the art the choice of whether to prepare a translucent or opaque composition.

For example, the presence of vegetable oils in the formulation generally gives the formulation an opaque appearance due to the nature in which it is dispersed. This demonstrates the ability of the formulations to suspend water insoluble or partially water soluble substances in the form of particles or liquid droplets therein.

All of the formulations A-L listed above are able to act as personal cleansing compositions which can incorporate insoluble benefit agents in a stable manner, have long shelf lives, are cost effective, and are easy to manufacture. Examples L-N already do contain insoluble benefit agents (visual effect pigment in Example L and sunflower oil in Examples M and N. Additionally, all these compositions have advantageous sensory qualities when used for personal washing, in particular producing a voluminous yet creamy lather and a pleasant skin-feel during the washing process which persists as a conditioned feel after washing.

It is of course to be understood that the present invention is not intended to be restricted to the foregoing example, which is described by way of illustration only.

1.-25. (canceled)

26. A post-foaming composition comprising:
a) water;
b) one or more anionic surfactants in an amount of about 2 to about 20% by weight of the composition;
c) one or more amine oxide surfactants in an amount of about 1 to about 10% by weight of the composition;
d) a hydrophobically modified alkali-swellable acrylic emulsion (HASE) polymer in an amount of about 0.5 to about 3% polymer solids by weight of the composition; and
e) a foaming agent comprising a non-polar saturated aliphatic hydrocarbon having from 3 to 6 carbon atoms;
wherein the composition is capable of suspending water insoluble or partially water soluble substances in the form of particles or liquid droplets.

27. A composition according to claim 26, wherein the HASE polymer is provided in the form of an aqueous liquid emulsion.

28. A composition according to claim 26, wherein the HASE polymer is selected from an acrylate/steareth-20 methacrylate copolymer, an acrylate/beheneth-25 methacrylate copolymer, an acrylate/steareth-20 methacrylate crosspolymer, and an acrylate/palmeth-25 acrylate crosspolymer.

29. A composition according to claim 26, wherein the HASE polymer is present in an amount of about 1 to about 2% by weight of the composition as polymer solids.

30. A composition according to claim 29, wherein the one or more anionic surfactants comprise one or more selected from alkyl sulfates; ethoxylated alkyl sulfates; alpha olefin sulfonates; alkyl alkoxy carboxylates; acyl isethionates; acyl sarcosinates; sulfosuccinates; or acyl glutamates;
wherein the one or more anionic surfactants comprise one or more selected from sodium lauryl sulfate, sodium cocosulfate, sodium laureth sulfate, sodium trideceth sulfate, sodium cocoyl isethionate, sodium cocoyl sarcosinate, disodium laureth sulfosuccinate, or sodium cocoyl glutamate.

31. A composition according to claim 30, wherein the one or more anionic surfactants comprise sodium laureth sulfate.

32. A composition according to claim 26, wherein the one or more anionic surfactants are present in an amount of about 5 to about 15% by weight of the composition.

33. A composition according to claim 26, wherein the one or more amine oxide surfactants comprise alkamine oxides and alkylamidopropyl amine oxides containing at least one hydrocarbon chain containing at least eight carbon atoms.

34. A composition according to claim 33, wherein the one or more amine oxide surfactants comprise one or more selected from alkyl dimethyl amine oxides of the formula: 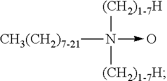
alkyl di(hydroxy lower alkyl)amine oxides of the formula: 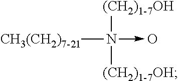
alkamidopropyl dimethyl amine oxides of the formula: 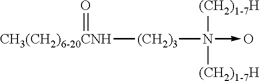
wherein the alkyl groups are straight or branched chain, saturated or unsaturated and contain from 8 to about 22 carbon atoms.

35. A composition according to claim 34, wherein the alkyl di(lower alkyl) amine oxides comprise one or more selected from lauryl dimethyl amine oxide, myristyl dimethyl amine oxide, dimethyl cocoamine oxide, myristyl/palmityl dimethyl amine oxide, myristyl/lauryl dimethyl amine oxide, cetyl dimethyl amine oxide, stearyl dimethyl amine oxide, and myristyl/cetyl dimethyl amine oxide;
wherein the alkyl di(hydroxy lower alkyl)amine oxides comprise one or more selected from bis(2-hydroxyethyl)cocoamine oxide, and bis(2-hydroxyethyl)stearylamine oxide; and
wherein the alkamidopropyl di(lower alkyl)amine oxides comprise cocoamidopropyl dimethyl amine oxide

36. A composition according to claim 26, wherein the one or more amine oxide surfactants comprise decyl dimethylamine oxide, lauryl dimethylamine oxide, stearyl dimethylamine oxide, oleyl dimethylamine oxide, coco dihydroxyethylamine oxide, cetyl N,N-dihydroxyethylamine oxide, oleyl N,N-dihydroxyethylamine oxide, cocamine oxide, cocamidopropylamine oxide, lauramidopropylamine oxide, oleamine oxide, oleamidopropylamine oxide, wheat germamidopropylamine oxide, isostearamido-propylamine oxide, stearamine oxide, stearamido-propylamine oxide, decylamine oxide, dihydroxyethyl C8-C10 alkoxypropylamine oxide, dihydroxyethyl stearamine oxide, myristamidopropylamine oxide, myristamine oxide, palmitamidopropylamine oxide, palmitamine oxide, undecylenamidopropylamine oxide, and mixtures of any two or more thereof.

37. A composition according to claim 26, wherein the one or more amine oxide surfactants comprise lauramine oxide, myristamine oxide cocamine oxide, lauramidopropyl amine oxide, myristamidopropyl amine oxide, cocamidopropyl amine oxide and mixtures of any two or more thereof.

38. A composition according to claim 26, wherein the one or more amine oxide surfactants are present in an amount of about 1 to about 5% by weight of the composition.

39. A composition according to claim 26, wherein the foaming agent comprises one or more compounds selected from iso-pentane, n-pentane, neo-pentane, iso-butane or n-butane;
wherein the foaming agent is optionally present in an amount of from about 2% to about 20% by weight of the composition.

40. A composition according to claim 26, wherein the pH of the invention is in the range of about 6.0 to about 8.0.

41. A composition according to claim 26, further comprising one or more ingredients selected from oils; natural or synthetic scrub particles; visual effect agents; humectants; cationic conditioning agents; preservatives, colorants, chelating agents, pH adjusters, or a clay-based rheology modifier; or
further comprising one or more ingredients selected from emollients, moisturizers, conditioners, skin conditioners, hair conditioners, vitamins, pro-vitamins, antioxidants, free-radical scavengers, abrasives, dyes, hair coloring agents, bleaching agents, UV absorbers, anti-UV agents, antibacterial agents, antifungal agents, melanin regulators, tanning accelerators, depigmenting agents, skin lightening agents, skin-coloring agents, liporegulators, weight-reduction agents, anti-acne agents, antiseborrhoeic agents, anti-ageing agents, anti-wrinkle agents, keratolytic agents, anti-inflammatory agents, anti-acne agents, antibiotics, anti-inflammatory agents, botanical extracts, imidazoles, refreshing agents, cicatrizing agents, vascular-protection agents, agents for the reduction of dandruff, seborrheic dermatitis, or psoriasis, shale oil and derivatives thereof, anti-psoriasis agents, corticosteroids depilating agents, agents for combating hair loss, reducing agents for permanent-waving, reflectants, essential oils and fragrances.

42. A method of manufacturing a personal cleansing composition according to claim 26, the method comprising the steps of:
i) loading an amount of water into a suitable vessel;
ii) adding a HASE polymer, one or more anionic surfactants, and one or more amine oxide surfactants, sequentially in any order, with sufficient mechanical stirring to ensure that each material is fully dispersed before adding the next;
iii) continuing mixing until a uniform consistency is achieved; and
iv) adding a foaming agent.

43. A method according to claim 42, the method comprising the steps of:
i) dissolving one or more anionic surfactants in an amount of water;
ii) adding a HASE polymer emulsion, mixing until the polymer is substantially dispersed in the one or more anionic surfactants;
iii) adding one or more amine oxide surfactants;
iv) mixing until a uniform consistency is achieved; and
v) adding a foaming agent.

44. A method according to claim 42, further comprising the step of packaging the composition into a container that can dispense a post-foaming gel.

45. A method of delivering one or more ingredients selected from emollients, moisturizers, conditioners, skin conditioners, hair conditioners, vitamins, pro-vitamins, antioxidants, free-radical scavengers, abrasives, dyes, hair coloring agents, bleaching agents, UV absorbers, anti-UV agents, antibacterial agents, antifungal agents, melanin regulators, tanning accelerators, depigmenting agents, skin lightening agents, skin-coloring agents, liporegulators, weight-reduction agents, anti-acne agents, antiseborrhoeic agents, anti-ageing agents, anti-wrinkle agents, keratolytic agents, anti-inflammatory agents, anti-acne agents, antibiotics, anti-inflammatory agents, botanical extracts, imidazoles, refreshing agents, cicatrizing agents, vascular-protection agents, agents for the reduction of dandruff, seborrheic dermatitis, or psoriasis, shale oil and derivatives thereof, anti-psoriasis agents, corticosteroids depilating agents, agents for combating hair loss, reducing agents for permanent-waving, reflectants, essential oils and fragrances to skin or hair, the method comprising applying a composition according to claim 1 to a body of a person.
